(19) United States
(12) Patent Application Publication
(10) Pub. No.: US 2009/0218243 A1
(43) Pub. Date: Sep. 3, 2009

(54) Packing for Injection Device

(75) Inventors: Steffen Gyrn, Ringsted (DK); Jens Egebjerg Nielsen, Ringsted (DK)

Correspondence Address: BRINKS HOFER GILSON & LIONE, P.O. BOX 10395, CHICAGO, IL, 60610, US

(73) Assignee: Unomedical A/S

(21) Appl. No.: 12/279,138

(22) PCT Filed: Feb. 13, 2006
(86) PCT No.: PCT/DK07/50018

§ 371 Date: Oct. 20, 2008

Related U.S. Application Data
(60) Provisional application No. 60/772,807, filed on Feb. 13, 2006.

(30) Foreign Application Priority Data
Feb. 13, 2006 (DK) PA 2006 00205

(51) Int. Cl. A61B 19/02

(52) U.S. Cl. 206/365

(57) ABSTRACT

This invention relates to a packing for an injector device for the placement of a subcutaneous infusion set on a patient. An insertion needle used in conjunction with an injector device is employed for transcutaneous placement of a soft and relatively flexible tubular cannula, followed by removal of the insertion needle and subsequent infusion of medical fluid according to the present invention can storage an injector device combined with an infusion set and an insertion needle under sterile conditions. The packing comprises at least—a first storage room storing the injector device combined with an infusion set and an insertion needle, —a first part (1) providing a further storage room (9b) isolated from the insertion needle (312, 26), is constructed with a bottom part (2a, 2b) and walls (2c) standing upright form the bottom part (2a, 2b), —a second part which is attached to the first part (1) before use in such a way that the conditions inside the packing remain sterile, and seen from a sectional view through the walls (2c), the walls (2c) are forming at least two sections each formed as a partial circle with at least two centres C1 and C2 and the centres C1 and C2 are placed with a distance D between them.

### BACKGROUND OF THE INVENTION

This invention relates to a packing for an injector device for the placement of a subcutaneous infusion set on a patient.

Medical needles are widely used in the course of patient treatment, particularly for delivery of selected medications. In one form, hollow hypodermic needles are employed for transcutaneous delivery of the medication from a syringe or the like, an insertion needle used in conjunction with an injector device is employed for transcutaneous placement of a soft and relatively flexible tubular cannula, followed by removal of the insertion needle and subsequent infusion of medical fluid to the patient through the cannula.

It is often necessary for a patient to transcutaneously place the medical needle himself. Diabetic patients for example frequently place a subcutaneous infusion set with a cannula for subsequent programmable delivery of insulin by means of a medication infusion pump.

Some patients are reluctant or hesitant to pierce their own skin with a medical needle, and thus encounter difficulties in correct needle placement for proper administration of the medication. Such difficulties can be attributable to insufficient manual skill to achieve proper needle placement or alternately to anxiety associated with anticipated discomfort as the needle pierces the skin. This problem can be especially significant with medications delivered via a subcutaneous infusion set, since incorrect placement can cause kinking of the cannula and resultant obstruction of medication flow to the patient. Cannula kinking can be due to infusion set placement at an incorrect angle relative to the patient's skin, and/or needle placement with an incorrect force and speed of insertion.

In relation to the known devices several different problems are recognized. Either the packing is compact and easy to handle but do not leave room for storage of extra equipment or accessories which is necessary or nice to have when applying the infusion set and protection of the insertion needle, or the packing can comprise extra equipment or accessories but is difficult to handle e.g. because it has a separate needle cover attached to the injector device which needle cover has to be removed before use.

The invention of the present application indicates a solution to these problems.

In order to provide the patient with a system comprising an injector device, an infusion set and necessary accessories such as tubing and connection (hub) for e.g. a pump or a reservoir which system can assure correct, easy and safe insertion of the infusion set, it is an advantage if the injector device combined with the infusion set and all other necessary components are delivered to the patient in one packing which is easy to gain access to and where the system is in a ready-to-use form making it uncomplicated for the patient to remove the injector from the sterile packing, connect the tubing of the infusion set to e.g. a pump or a reservoir and inject the infusion device, without having to interconnect any components of the system whether that could be attaching the infusion set to the injector or connecting the tubing to the infusion part.

An example of injector devices which can be enclosed in the packing is disclosed in WO03/026728, incorporated by reference herein.

The present invention is aimed at providing a packing for an injector device, which allows for protecting the sharp-pointed needle which is used to penetrate the patient's skin and allows for including tubing and large or heavy pieces such as a hub beside the injection device inside the packing. The present invention also aims at providing a packing which allows for the injection device to take at least two positions inside the packing, in a first position the injection device is secured to the packing and in a second position it is possible to remove the injection device from the packing.

### SUMMARY OF THE INVENTION

The invention concern a packing inside which an injector device combined with an infusion set and at least one insertion needle can be kept under sterile conditions which packing comprises at least
a first part made of a material which can not be penetrated by an insertion needle,
a second part which is attached to the first part before use in such a way that the conditions inside the packing remains sterile,
a first storage room storing the injector device combined with an infusion set and an insertion needle and characterized in that the first part provides a further storage room isolated from the insertion needle.

The storage room is an open room defined by the walls of the first part of the packing and by a surface of the combined injector device. The extra storage room can be used for keeping equipment such as fittings for external equipment, connectors attached to the tubing from the infusion device etc. under sterile conditions, while at the same time protecting the insertion needle which will normally be <0.5 mm in outer diameter, preferably <0.3 mm in outer diameter. These very thin insertion needles are normally used when insertion is performed with an injector device as the injector device assures that the insertion needle penetrates the skin of the patient in a correct angle without twisting or bending the insertion needle.

In one embodiment of the invention the further storage room is adjacent to the proximal side of the infusion set and the further storage room has at least one wall provided by a needle cover extending from the inner surface of the first part toward the proximal side of the infusion set thereby isolating the insertion needle. In this embodiment the needle cover is integrated with the cover isolating the needle/cannula side of the injector device from the surroundings.

In a second embodiment of the invention the further storage room is adjacent to a non-proximal side of the insertion device. A non-proximal side is a distal side of the injector device combined with the infusion set and the at least one insertion needle. In this second embodiment there is no needle cover isolating the needle/cannula side of the injector device from the surroundings, the further storage room is formed by the first part of the packing and e.g. a distal surface of the combined injector device.

In the second embodiment of the invention the further storage room is preferably adapted for at least partly holding the injection device after use, this can be done by providing the further storage room with restrictions which restrictions will secure the injection device to the inside of the first packing after use.

Preferably the first part of the packing is constructed with a bottom part and walls standing upright form the bottom part and forming a rim opposite the bottom part and the second part comprises one piece of material which can be secured to the rim.

In a preferred embodiment the walls, seen from a sectional view through upright standing material, form at least two sections each formed as a partial circle with at least two centres C1 and C2 and the centres C1 and C2 are placed with a distance D between them. Preferably the radius of the two partial circles, R1 and R2, are not identical, R2<R1.

In a preferred embodiment the section with the centre C1 has a radius R1 large enough to hold the injector device without restricting removal of the device from the packing, and preferably this section should be large enough to hold the injector device wrapped with at least one layer of infusion tubing.

In a specially preferred embodiment the section with the centre C2 has a radius R2 large enough to hold the housing of the injector device, and preferably the section with the centre C2 has restrictions which secure the injector device to the first part of the packing. These restrictions should prevent the used injection device to move out of the first part of the packing in a direction parallel with the walls of the first part of the packing. Also such restrictions could prevent the used injection device to move between the section with centre C1 and the section with centre C2.

The invention also concerns a combined injector device comprising an infusion set, at least one insertion needle, a housing, the injection device is releasably connected to the infusion set and the infusion set is connected to an infusion tubing, where the infusion tubing is placed outside the housing of the injector device during storage under sterile conditions. As it is preferred to remove the tubing from the packing before the injection device is removed from the clean packing, it is more efficient to place the tubing outside the housing of the injection device as this makes the tubing accessible. Preferably the infusion tubing is coiled around the outer surface of the housing during storage.

In a more preferred embodiment the invention concerns an injector device combined with an infusion set and an insertion needle which combination before use is kept under sterile conditions in a packing comprising at least
a first part made of a material which can not be penetrated by an insertion needle,
a second part which is attached to the first part before use in such a way that the conditions inside the packing remains sterile,
and the injector device comprises a housing and is releasably connected the infusion set which infusion set is connected to an infusion tubing,
   characterized in that the infusion tubing is placed between the outer surface of the housing of the injector device and the inner surface of a first part of the packing during storage.

In a more preferred embodiment the invention concerns an injector device assembly for transcutaneously placing a hollow cannula of a subcutaneous infusion set through the skin of a patient where the injector device is releasably connected to the infusion set (14) during storage, and where the injector device comprises:
a device housing,
a plunger slidably received within the device housing for movement between an advanced position and a retracted position, an insertion needle is either secured to the plunger for receiving and supporting the cannula of the subcutaneous infusion set or insertion needle is constituted by the cannula, the infusion set, which is releasably connected to the plunger, is in a position oriented for transcutaneous placement of the cannula upon movement of the plunger from the retracted position to the advanced position,
a drive for urging the plunger from the retracted position toward the advanced position to transcutaneously place said cannula of said subcutaneous infusion set received on said insertion needle,
   and the infusion set comprises:
a housing connected to an infusion tubing by a suitable connector, wherein the infusion tubing is positioned close to the outer surface of the housing of the injector device during storage, and preferably the infusion tubing is coiled wholly or partly around the housing of the injector device, and more preferred the outer surface of the housing is provided with guiding or positioning means for the tubing.

One purpose of the packing according to the present invention is to form a closed shell around the injector and the infusion set in order to prevent the device from being polluted with micro organisms. A second purpose is to protect the injection needle, which could be the cannula, from impacts from the surroundings as the cannula/injection needle is very thin and delicate, and also to protect the surroundings from the injection needle, especially when the insertion needle has been used and has to be disposed of. A third purpose is to make it possible to include a whole system for injecting an infusion set and connecting this set to a device such as a pump or a reservoir in a packing in a ready-to-use state.

### BRIEF DESCRIPTION OF THE DRAWINGS

The accompanying drawings illustrate the invention.

FIG. 1 is a perspective view of a known infusion device suitable for use with an injector device, and

FIG. 2 shows in an exploded view a known embodiment of an injector device assembly wherein the plunger has an insertion needle secured thereto,

FIGS. 3a and 3b show in a perspective view the known injector device of FIG. 1 with the plunger in the advanced position,

FIGS. 4a and 4b show in a perspective view the injector device of FIG. 2 with the plunger in the retracted position,

FIGS. 4c-4e show views similar to FIGS. 3a, 4a and 4b with part of the housing being cut away,

FIGS. 5 A and B show respectively a view of the inner surface and a view of the outer surface of a first part of a packing of one embodiment according to the invention,

FIG. 6 shows a view of a first part of a packing of a second embodiment according to the invention,

FIG. 7 shows a three-dimensional view of the second embodiment of FIG. 6,

FIG. 8 shows a housing of an injector device according to the invention.

### DETAILED DESCRIPTION OF THE PREFERRED EMBODIMENTS

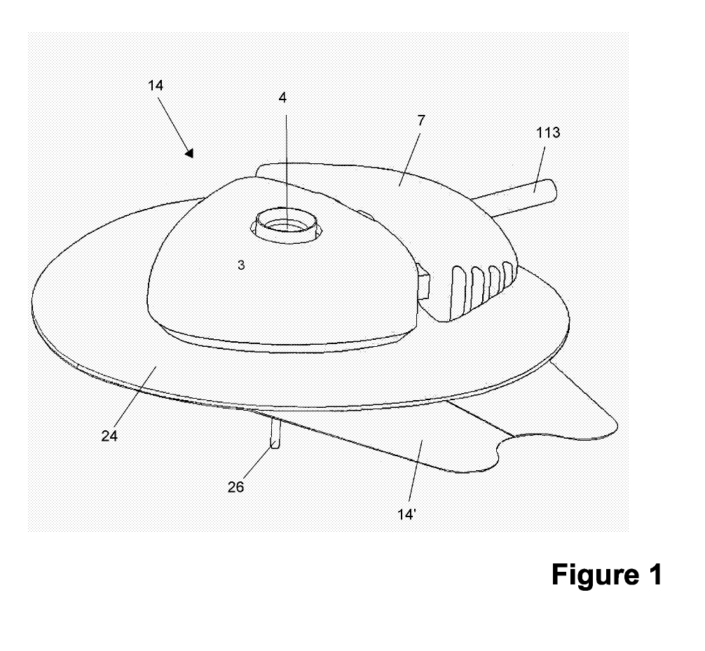

FIG. 1 shows an example of an infusion set 14 suitable for use with an injector device. The infusion set 14 includes a housing 3 with an internal chamber (not shown). The internal chamber receives medication via infusion tubing 113 which may be detachably connected to the housing 3 by any suitable connector 7. The base 24 of the housing 3 may be a flexible sheet of a woven material secured to the housing 3 such as by means of an adhesive and carrying an adhesive covered by a release sheet 14′ which is removed to expose the adhesive prior to placement of the infusion set. The infusion set 14 has a protruding soft and flexible cannula 26, which communicates with the internal chamber. An internal passage which is sealed by a sealing membrane 4 and which is penetrated by the insertion needle of the injector device extends through the housing opposite the cannula 26.

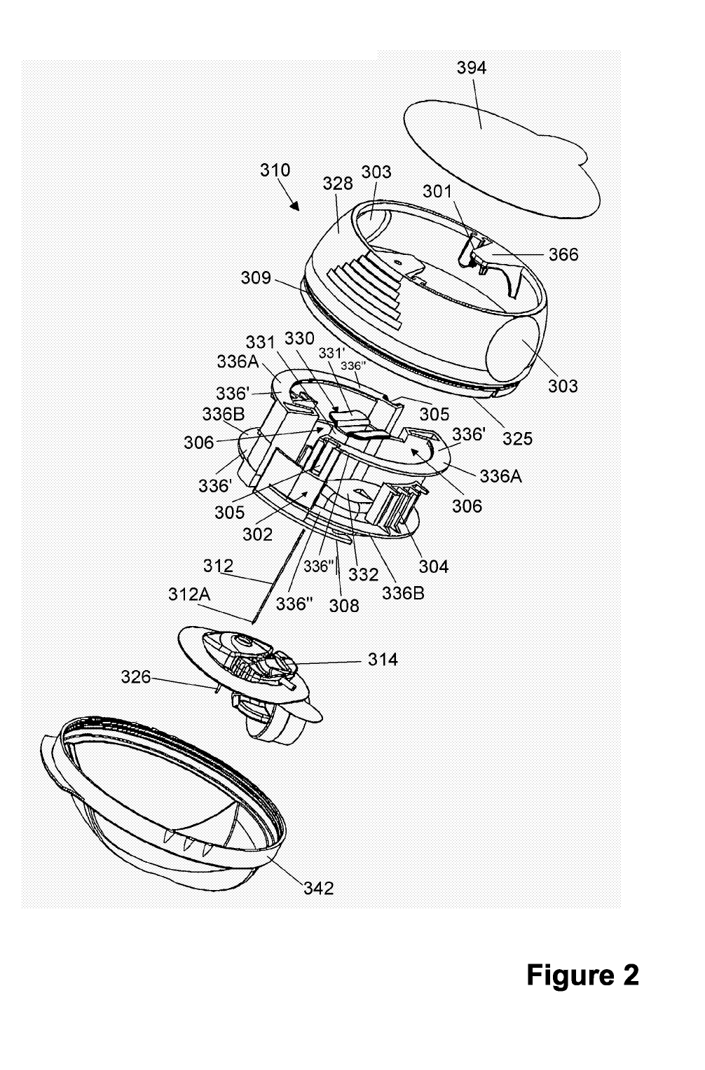

FIG. 2 shows in an exploded view a known embodiment of an injector device assembly.

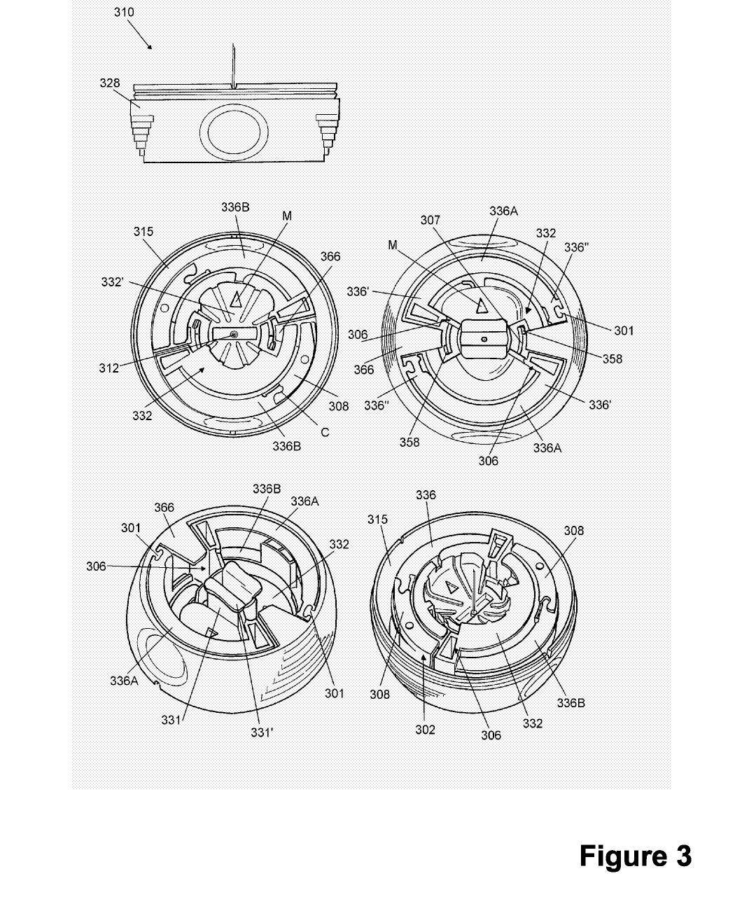

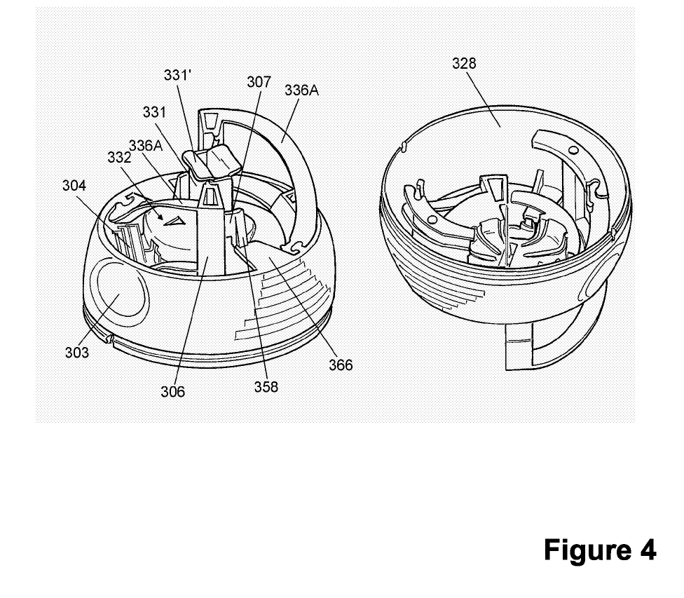

FIGS. 3 and 4 show the same embodiment in different views and positions.

The packing of the injector device 310 includes a housing 328 and respective removable covers 342, 394. The cover 342 has a hollow for accommodating a part of an insertion needle 312 when the cover 342 is secured to the housing 328, such as by snap engagement with the rim 309 of the housing 328. The cover 342, the housing 328, a plunger 330 and a drive with a spring for advancing the plunger 330 to the advanced position can be made of plastics while the cover 394 may be a flexible foil secured to the housing 328 by an adhesive. Preferably, the covers 342, 394 serve as bacterial barriers, the flexible foil 394 being of medical paper. An insertion needle 312 is preferably secured in a stable manner to the plunger 330 of the injection device, such as by press-fitting, the plunger 330 having a narrow central passage wherein an end of the insertion needle 112 is lodged. The plunger 330 and the drive may be formed integrally as a single component in a molding process.

The ring-shaped housing 328 is flexible in the sense that the application of a manual force against diametrically opposed depressions 303 of fingertip size will give rise to a slight deformation of the housing 328 such that it assumes a slightly oval shape when viewed from above for bringing about a release of the plunger in the retracted position and cause a spring-loaded movement of the plunger 330 towards the advanced position, as will be explained. For maintaining the plunger 330 in the retracted position the housing 328 is provided with two opposed ledges 366. Moreover, the housing 328 is provided with opposed dovetail projections 301 extending along the same general direction as the insertion needle 312 and adapted to connect with complementary recesses in the aforementioned spring, to secure the spring in relation to the housing 328.

The plunger 330 generally includes a head 332, a hub 331 and, opposite the head 332, an enlarged gripping portion 331′ which allows a user to manually pull the plunger 330 to a retracted position. The head 332 normally carries a marking M representing the place where the 113 tubing exits the infusion set 314 located there under whereby the user can check the orientation of the tubing after placement of the infusion set. The head 332 moreover has a recess 332′ for accommodating the infusion set 326 with cannula 326 through which the insertion needle 312 extends, the infusion set 314 preferably being maintained in position by frictional engagement of the insertion needle 312 with an inside surface of the infusion set 314. The plunger 330 has two opposed rigid walls 306 extending radially outwardly from the hub 331. The walls 306 extend in the axial direction of the device 310, i.e. in the same general direction as the insertion needle 312, and are connected to the aforementioned spring. Moreover, as best seen in FIG. 3d, the walls 306 each carry a lateral projection 307 with a finger 358 which is releasably locked in engagement with a corresponding one of the ledges 366 of the housing 328 by snap action in the retracted position of the plunger 330. The depressions 303 preferably being offset with respect to the ledges 366 by about 90° will cause the opposed ledges 366 to move apart when the aforementioned manual force is applied and the housing 328 assumes an oval shape, thereby bringing the finger 358 on each wall 306 out of engagement with the corresponding ledge 366. For retaining a proximal part of the tubing 113 (not shown) which is wound around the plunger 330, wall 306 has a groove G best seen in FIGS. 4c and 4d sized to receive a small length of the tubing 113 and to prevent the infusion set 314 from being inadvertently pulled away from the plunger 330 by the user when the tubing is unwound for connection with a medical fluid supply.

The drive which acts to drive the plunger 330 from the retracted position towards the advanced position when the fingers 358 are disengaged comprises a spring including four thin and flexible plastics strips, of which two opposed strips 336A extend about halfway around the plunger 330 at the level of the gripping portion 331′ while two other opposed strips 336B extend about halfway around the plunger 330 at the level of the head 332, as viewed in the advanced and unbiased position of the plunger shown in FIGS. 2 and 3a-e. One end 336′ of one of the strips 336A and one end 336′ of one of the strips 336B is rigidly connected to one of the walls 306, while one end 336′ of the other one of the strips 336A and one end 336′ of the other one of the strips 336B is rigidly connected to the other one of the walls 306. Preferably, the strips 336A and 336B are integrally connected with the walls 306 in a molding process where the plunger 330 and the spring formed from the strips 336A and 336B is formed in one molding operation.

The spring also comprises two rigid opposed rigid walls 302 that extend in the axial direction of the device 310 and that are each rigidly connected with the second end 336″ of one of the strips 336A and the second end 336″ of one of the other strips 336B. The rigid walls 302 are preferably integrally connected with the strips 336A and 336B at the second end thereof. The walls rigid 302 each have an axially extending recess 305 which is complementary with the dovetail projection 301 on the housing 328. When the plunger 330 with the spring is mounted within the housing 328 the dovetail projection 301 is slid into the recess 305 by axial movement; by selecting proper dimensions of the dovetail projection 301, and possibly also by performing this operation at a predetermined temperature, a press-fit may result that prevents subsequent removal of the plunger 330. Alternatively, or additionally, the plunger 330 may be secured using glue, or using a welding process. The two rigid walls 302 of the spring also comprise a respective projection 308 with a lower surface which in the advanced position of the plunger 330 is essentially coplanar with the rim 309 of the housing 328. The projections 308 include a clip-like retainer C for securing a distal part of the tubing 113 wound around the plunger 330, thereby maintaining the tubing in position until unwound by the user.

As will be understood, the walls 302 are fixed in relation to the housing 328, and the strips 336A and 336B, being thin and flexible, define the parts of the spring that undergo a change in shape upon retraction of the plunger 330 and that through this change of shape generate the force acting on the plunger 330 via the connections at the ends 336′ and required to advance the plunger 330 to the advanced position upon disengagement of the fingers 358. The shape of the strips 336A and 336B in the deformed condition when the plunger 330 is held in the retracted position is shown in FIGS. 4a-d. The connection between the strips 336A, 336B and the walls 302, 306 being rigid, in the sense that bending moments arising in the strips 336A, 336B upon retraction of the plunger 330 are transferred to the walls 302, 306, brings about a deformation of the strips 336A, 336B as shown.

It will be understood that the resiliency of the spring is generally defined by the elastic properties of the flexible strips 336A, 336B which should be selected such that the drive is capable of advancing the plunger 330 to the advanced position at least once, following retraction. The spring would normally allow the piston to be retracted several times, and provide the required force for subsequently advancing the plunger 330. However, the device being normally a disposable unit requires the spring to be formed with the capability to only a limited number of times advance the plunger 330 at one given speed, and the spring need not be capable of returning the plunger to the exact original position after several times of use.

As seen best in FIG. 2, the two strips 336B each carry a wall member 304 which provides support for a tubing (not shown) connected to the infusion set 314 and wound around the plunger 330 in the annular space 315 between the plunger 330 and the housing 328.

In this embodiment the housing 328 constitutes the packing and this necessitates that the tubing 113 is wound around on the inside of the housing 328 in order for the tubing to be protected by the packing.

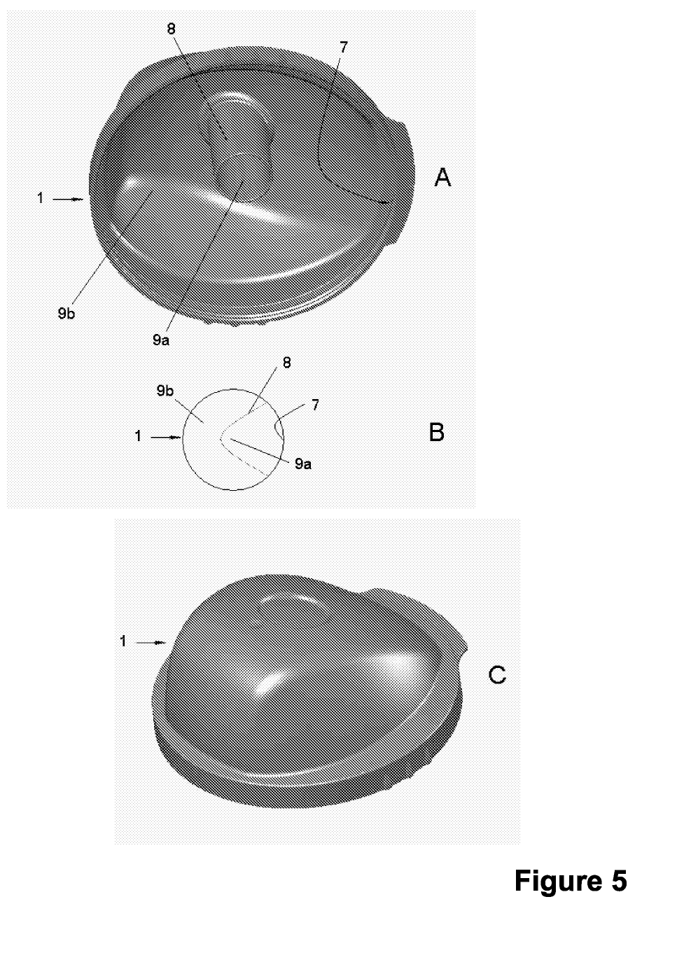

FIGS. 5 A and B shows an embodiment of a first part 1 of the packing according to the invention seen from the side being adjacent to the insertion needle, this embodiment has one storage room which isolates the insertion needle 9a and one storage room for accessories 9b. In this embodiment the first part 1 replaces the removable cover 342 of the known injection device and the second part is constituted by the housing 328 and the second removable cover 394. The cover 342 is made of a relatively hard material and has a hollow for accommodating the insertion needle 312 when the cover 342 is secured to the housing 328, but the cover is only intended to protect the insertion needle 312 from impacts and actions coming from the outside of the packing. In order to protect the delicate insertion needle 312 from actions coming from the inside of the packing, e.g. actions origination from accessories to the combined injection system laying unsecured in the sterile storage room next to the insertion needle 312, the first part 1 is provided with a needle cover 8 extending from the inner surface 7 of the first part 1 and completely surrounding the insertion needle 312. In this embodiment the second storage room 9b which is isolated from the insertion needle 312 has the form of a circular band with a vacant circular centre in which the insertion needle 312, 26 is positioned when the first part 1 of the packing is joined to the injector device 310, but the needle cover 8 could also have the form of a wall being connected at two positions to the inner surface 7 of the first part 1 of the packing as illustrated in FIG. 5 B.

The needle cover 8 is preferably made of a continuous sheet of material providing a continuous protective wall for the insertion needle 312 but the needle cover 8 can be made of a material different from the first part 1 of the packing and the needle cover 8 can also be made as a non-continuous wall e.g. be made of upright standing posts or the like which provides for a non-continuous wall but although non-continuous the wall continues to protect the insertion needle 312 against the unit or units being stored between the inner walls 7 of the first part 1 of the packing and the insertion needle 312 as long as the openings in the needle cover 8 are small enough to prevent contact between the unsecured unit(s)/accessories and the insertion needle 312.

FIG. 5 C shows the first part 1 of the packing seen from the outer side i.e. the non-sterile side of the packing.

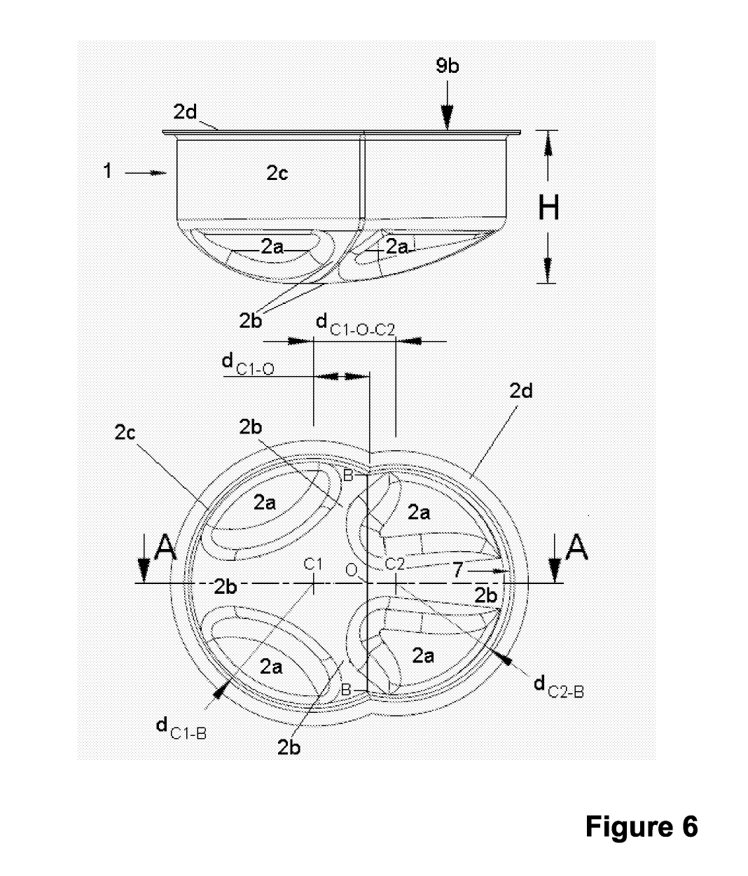

FIG. 6 shows a first part 1 of a packing according to the invention, the first part 1 of the packing consist of a rim 2d and a shaped hollow comprising a bottom part 2a, 2b and a wall part 2c with an inner surface 7. In order to provide the packing with an adequate steadiness, the bottom part is preferably constituted with a plurality of hollow 2a and elevated 2b areas. In FIG. 5 the bottom part is provided with four hollows 2a forming a cross-like elevated part 2b. The elevated part 2b extends along the line A-A and along the lines from C1-B on both sides of the rim 2d.

The first part 1 of the packing covers the cannula side of the injection device 310, 310′ inside the packing and is made of a relatively hard material such as polypropylene (PP) or polyethylene (PE) or another material which cannot be penetrated by the injection needle. The relatively hard material will protect the injection needle against impacts from the surroundings and also the surroundings will be protected against the injection needle 312. The injection needle can either be a sharp needle 312 unreleasably connected to the injector device 310, 310′ or it can be the cannula 26, 326 of the infusion set 14 when the cannula is constructed of a hard material. A second part of the packing (not shown) of this embodiment covers the opening of the first part 1 of the packing which opening is formed of the rim 2d and turned away from the injection needle 312, 26. This means that the second part of the packing does not need to protect the insertion needle and can be made of a soft material which is e.g. glued or welded to the rim 2d of the first part 1 of the packing.

When seen from the rim side, which will also be referred to as the top side, the packing of this embodiment has the form of two partial circles with different diameter, D1 and D2. The two circles are larger than half their full size which means that the line B-B where they meet forms the narrowest part of the shape formed by the rim 2d. No matter which forms the two sections may have it will be preferred to provide the space shaped by the walls 2c with a reduced cross-section indicated with a line (B-B) in FIG. 5. The center of the largest partial circle is marked with C1 and the center of the smallest partial circle is marked with C2 and the position where the line B-B crosses the line A-A is marked with O. The line B-B will in this embodiment always be perpendicular to the line A-A and cross the line A-A at a position between the two center markings C1 and C2. The distance D between the two center markings C1 and C2 is in the figure named d_{C1-O-C2}.

In this embodiment the distance between the inner surface of the walls 2c at the line B-B is almost the same as the outer diameter of the housing 328 of the injector device 310, 310′, preferably the distance between the inner surface of the walls 2c at line B-B is slightly smaller than the housing 328 of the injector device and the walls 2c have a certain flexibility which will make it possible to force the housing 328 of the injector device 310, 310′ from the circle part with the largest diameter to the circle part with the smallest diameter and then lock the injector device 310, 310′ in this position as the flexibility of the walls 2c of the packing will prevent the injector device from slipping back into the circle part with the largest diameter.

In a preferred embodiment the device has the following measures:

Outer radius of the housing 328 incl. guiding means 5=57 mm
Outer radius of the housing 328 excl. guiding means 5=55 mm
d_{C1-B}=R1=30.2 mm
d_{C2-B}=R2=27.7 mm
D=d_{C1-O-C2}=20.0 mm
d_{C1-O}=13.74 mm
d_{B-O}=\sqrt{30.2²−13.74²}=26.89 mm [symbol]
d_{B-B}=2*d_{B-O}=53.77 mm (distance between inner walls at line B-B)

The first part 1 of the packing can be provided with means for locking the injector device 310, 310′ to the inside of the packing of the circle part with the smallest diameter. This can be done in a simple way by extending the rim 2d of the circle part with the smallest diameter either partly, i.e. by forming protrusions extending inwardly from the rim 2d toward the center C2, or as a whole i.e. the whole rim is extended toward the center C2 thereby decreasing the diameter of the partial circle part at the rim 2d level. Which solution is the most appropriate would depend on the material used to make the first part 1 of the packing and the rim 2d of the packing, generally the more stiff and steady the material is the fewer protrusions or the smaller protrusion area will be needed to detain the injector device inside the packing.

The height H representing the total height of the first part 1 of the packing comprising both the walls 2c and the bottom part 2a, 2b should be deep enough to surround and protect the insertion needle.

The area of the packing placed closest to—and facing—the insertion needle, in this embodiment the central part of the packing along the line A-A, will have a height H sufficient to enclose and protect the insertion needle whether the injection device is placed in the partial circle with the smallest or the largest diameter.

FIG. 6 shows a three-dimensional view of the embodiment from FIG. 5.

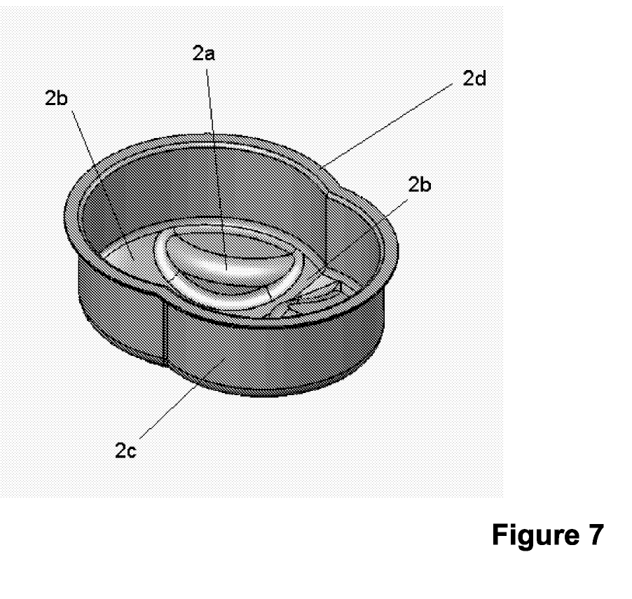

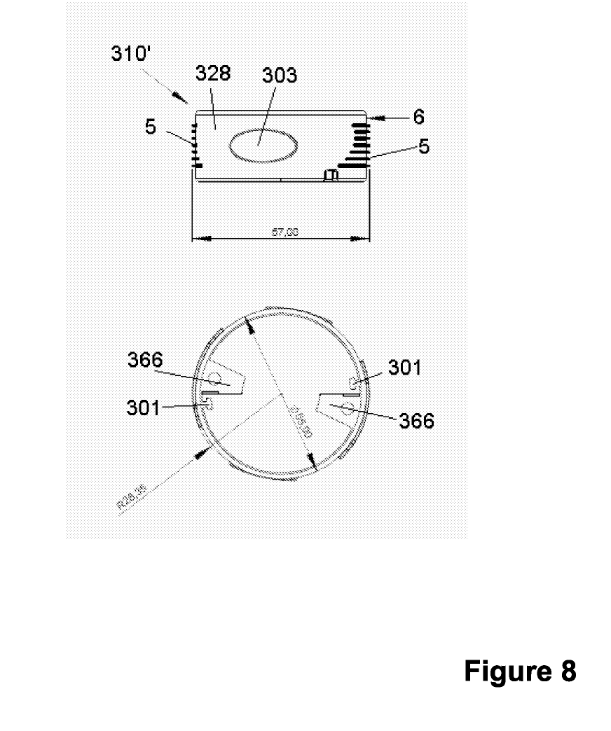

FIG. 7 shows an embodiment of an injection device which can be packed in the embodiment of the packing described in FIGS. 5 and 6. In this embodiment guiding means 5 are placed on the outer surface 6 of the housing 328.

Like the known device shown if FIG. 2-4 the injection device 310′ comprise a ring-shaped housing 328 which is flexible in the sense that the application of a manual force against diametrically opposed depressions 303 of fingertip size (Only one is shown) will give rise to a slight deformation of the housing 328 such that it assumes a slightly oval shape when viewed from above for bringing about a release of a plunger in the retracted position and cause a spring-loaded movement of the plunger towards an advanced position. For maintaining the plunger in the retracted position the housing 328 is provided with two opposed ledges 366. The housing 328 is also provided with opposed dovetail projections 301 extending along the same general direction as the insertion needle and adapted to connect with complementary recesses in the spring, to secure the spring in relation to the housing 328. The plunger can be as described above and shown in FIGS. 2, 3 and 4.

As the packing will isolate the injector device 310′ from the surroundings it is not necessary to keep the tube 113 inside the housing 328 before use, and the injector device is provided with horizontal flanges 5 which can keep the coiled tube 113 in place when the injector device is placed inside the packing.

Before use and during storage the injector device 310′ is kept inside the packing, the needle/cannula side of the injector device 310′ is turned towards the first part of the packing and a second part of packing is secured to the rim 2d of the first part of the packing in order to assure an airtight closure of the sterile packing. The injector device 310′ is placed in the circle part with the largest diameter and the center C1, the tube 113 is coiled around the injector device 310′ and fitted in between the flanges 5, the connector (not shown) which is unreleasably fastened to the tube 113 and which can connect the tube to e.g. a pump and/or a reservoir for medication is placed in the circle part with the smallest diameter.

When the user wants to insert an infusion set 14 to the skin the following steps are performed:
I. The second part of the packing is removed.
   Preferably the second part (not shown) of the packing has the form of a flexible membrane made by e.g. paper or plastic being glued or molded to the rim 2d of the first part 1 of the packing.
II. The user take hold of the connector placed in the circle part with the smallest diameter, unwind the tube 113 which is coiled around the injector device 310′ and connects the tube 113 to a device that can provide fluid through the tube 113 e.g. to a pump combined with a reservoir.
III. After unwinding the tube 113 it will be easy for the user to lift the injector device 310′ out of the first part 1 of the packing, bring the plunger to the retracted position, place the injector device 310′ against the skin and press the diametrically opposed depressions 303 thereby forcing the plunger to a forward position and inserting the infusion set 14. The infusion set 14 is left inserted in the patient's skin while the injector device 310′ is removed.
IV. After use the injector device 310′ is replace in the first part 1 of the packing in the circle part with the largest diameter, and from there the injector device 310′ is pushed into the circle part with the smallest diameter. Preferably the circle part with the smallest diameter is provided with means for retaining the injector device inside the first part 1 of the packing which will make it possible to dispose of the injector device after use without having to think about how to prevent surroundings from being exposed to the infected needle of the injector device 310′.

In order to make it possible to place the injector device 310′ inside the first part of the packing it is necessary that the outer dimension of the injector device, preferably the outer dimensions of the injector device 310′ with the tube 113 coiled around it, is smaller than the inner dimension of at least a part of the first part 1 of the packing, preferably the inner dimension of the circle part with the largest diameter.

In order to fasten the injector device 310′ inside the packing after use, at least a part of the packing is provided with a restricted room. In one embodiment this restricted room is partly constructed of the circle part with the smallest diameter and the center C2. The restriction can comprise a combination of a reduced cross-section e.g. as formed at the line B-B and one or more protrusions extending inward at the rim level.

1. A packing inside which an injector device combined with an infusion set and an insertion needle can be kept under sterile conditions the packing comprising:
a first storage room in a first part, the first storage room configured to store the injector device combined with an infusion set and an insertion needle,
a further storage room provided in the first part isolated from the insertion needle and constructed from a bottom part and walls standing substantially upright from the bottom part the walls forming at least two sections each formed as a partial circle with at least two centres C1 and C2 and the centres C1 and C2 are different and placed with a distance D between them which allows for the injection device to take at least two positions inside the packing, and
a second part attached to the first part before use in such a way that the conditions inside the packing remain sterile.

2. A packing according to claim 1, wherein the injection device has a first position wherein the injection device is secured to the packing and a second position wherein the injection device can be removed from the packing.

3. A packing according to claim 2, wherein the further storage room comprises restrictions configured to secure the injection device the first part after use so that the injection needle is covered.

4. A packing according to claim 3, wherein the restrictions are provided by extending the rime of the further storage room inward.

5. A packing according to claim 3, wherein the distance between the an inner surface of the walls at a line where the two partial circles meet is slightly smaller than a housing of the injector device and the walls are configure to flex to force the housing of the injector device from first position to the second position and then lock the injector device in this second position so that the flexible walls of the packing will prevent the injector device from slipping back into the first position.

6. A packing according to claim 2, wherein the further storage room is adapted for at least partly holding the injection device after use.

7. A packing according to claim 1, wherein the first part comprises a material which can not be penetrated by an insertion needle.

8. A packing according to claim 1, wherein a rim is formed on the walls opposite the bottom part and the second part comprises a connecting portion that can be secured to the rim.

9. A packing according to claim 1, wherein a radius of one partial circle is not identical to the other partial circle.

10. A packing according to claim 6, wherein the section with the centre C1 has a radius R1 large enough to hold the injector device without restricting removal of the device from the packing.

11. A packing according to claim 6, wherein the section with the centre C2 has a radius R2 large enough to hold a housing of the injector device.

12. A packing according to claim 8, wherein the section with the centre C2 has restrictions which secure the injector device to the first part of the packing.

13. The packing according to claim 4, wherein the rim extends inward by forming protrusions from the rim toward a center or by extending the whole rim toward the center to decrease the diameter of at least one of the partial circles.

14. A packing for an injector device combined with an infusion set and an insertion needle, the packing assuring sterility of the injector device, infusion set and insertion needle therein prior to use of the injector device, the packing comprising:
a first portion having a bottom wall and at least one upstanding side wall, the first portion comprising:
a first area defined in the first portion to house at least a portion of the injector device combined with an infusion set and an insertion needle;
a second area defined in the first portion and isolated from the insertion needle, the second area comprising at least two sections of different sizes, each section formed as a partial circle having a center, the centers being spaced apart so that the injection device can be positioned in the first portion in two different positions inside the packing; and
a second portion removably attached to the first portion so that the conditions inside the packing remain sterile before use of the injection device.

15. the packing according to claim 14, wherein the second area is adapted for at least partly holding the injection device after use.

16. The packing according to claim 14, wherein the packing is sized and shaped to include tubing connected to the injector device.

17. The packing according to claim 14, wherein one section having a radius R1 is large enough to hold the injector device without restricting removal of the device from the packing.

18. The packing according to claim 17, wherein the other section having a radius R2 is enough to hold a housing of the injector device.
